(19) United States
(12) Patent Application Publication
(10) Pub. No.: US 2024/0099627 A1
(43) Pub. Date: Mar. 28, 2024

(54) FORCE ESTIMATION FROM WRIST ELECTROMYOGRAPHY

(71) Applicant: Apple Inc., Cupertino, CA (US)

(72) Inventors: Matthias R. HOHMANN, Mountain View, CA (US); Ellen L. ZIPPI, Daly City, CA (US); Kaan E. DOGRUSOZ, San Francisco, CA (US)

(21) Appl. No.: 18/369,835

(22) Filed: Sep. 18, 2023

Related U.S. Application Data
(60) Provisional application No. 63/408,467, filed on Sep. 20, 2022.

(51) Int. Cl. A61B 5/22; A61B 5/00; A61B 5/389

(52) U.S. Cl. CPC A61B 5/224; A61B 5/389; A61B 5/681; A61B 5/7225

(57) ABSTRACT

Aspects of the subject technology provide improved techniques for estimating muscular force. The improved techniques may include single-channel or multiple-channel surface electromyography (EMG), such as via a measurement device worn on a wrist. A muscular force estimate may be based on one or more measurements of variation between adjacent voltage measurements and estimates of spectral properties of the voltage measurements. The resulting muscular force estimate may for a basis for improved hand gesture recognition and/or heath metrics of the user.

The present application claims the benefit of U.S. Provisional application, Ser. No. 63/408,467 filed Sep. 20, 2022, entitled “FORCE ESTIMATION FROM WRIST ELECTROMYOGRAPHY.” The aforementioned application is incorporated herein by reference in its entirety.

### TECHNICAL FIELD

The present description relates generally to measurements of muscular force and gesture recognition.

### BACKGROUND

Surface electromyography (EMG) generally involves placing several electrodes scattered around an area of the skin of a subject in order to measure electrical potential (voltage) across nerves or muscles of the subject.

### BRIEF DESCRIPTION OF THE DRAWINGS

Certain features of the subject technology are set forth in the appended claims. However, for purpose of explanation, several implementations of the subject technology are set forth in the following figures.

FIG. 1 illustrates an example system for gesture recognition.

FIG. 2 illustrates an example system for measuring muscular force.

FIG. 3 illustrates an example process for estimating muscular force.

FIG. 4 illustrates an example process and system for probabilistic gesture control in accordance with one or more implementations.

FIG. 5 illustrates an example system for measuring muscular force.

FIG. 6 illustrates an example process for estimating muscular force.

FIG. 7 illustrates a perspective view of an example electronic device in accordance with one or more implementations.

FIG. 8 illustrates an example computing device with which aspects of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Techniques are presented for improved muscular force estimates. The improved techniques may include single-channel or multiple-channel electromyography (EMG), where EMG measurements are taken with electrodes such as via a measurement device worn on a wrist. A resulting muscular force estimate may be used, for example, for improving hand gesture recognition and/or for producing a health metric for a user. Electrodes may provide a series of voltage measurements over time of a subject user, from which a muscular force may be estimated. In an aspect, the estimate may be based on the measurements of a differential pair of electrodes.

In some implementations, the estimate of muscular force may be based on one or more of measures derived from EMG voltage measurements. For example, the estimate of muscular force may be based on a measure of variation between adjacent voltage measurements (e.g., standard deviation of differences between adjacent voltage measurements (DASDV), or median absolute deviation (MAD)). In a second example, the estimate of muscular force may be based on estimated spectral properties of the voltage measurements, such as a spectral moment. In a third example, the muscular force estimate may be based on a combination of measures of variation, spectral properties, and/or other measurements such as fractal dimension metrics or derivation-based metrics, which will collectively be referred to as “stability” metrics in this application.

In other implementations, the estimate of muscular force may be based on an estimated mean frequency of the voltage measurements, such as a first-order spectral moment calculated from the voltage measurements. In some aspects, an estimate of muscular force for a user may be adjusted based on calibration information derived from a calibration process with that particular user.

An estimate of muscular force may be used to improve gesture recognition. In an aspect, an EMG device may be attached to a subject user's wrist for generating voltage measurements related to muscular forces of the user's hand. In another aspect, a separate sensor for recognizing gestures of the user's hand, such as a camera for capturing images of the hand, may detect gestures of the hand. In one aspect for improved gesture recognition, a muscular force estimate from an EMG device may be used to adjust a preliminary confidence estimate of a detected gesture.

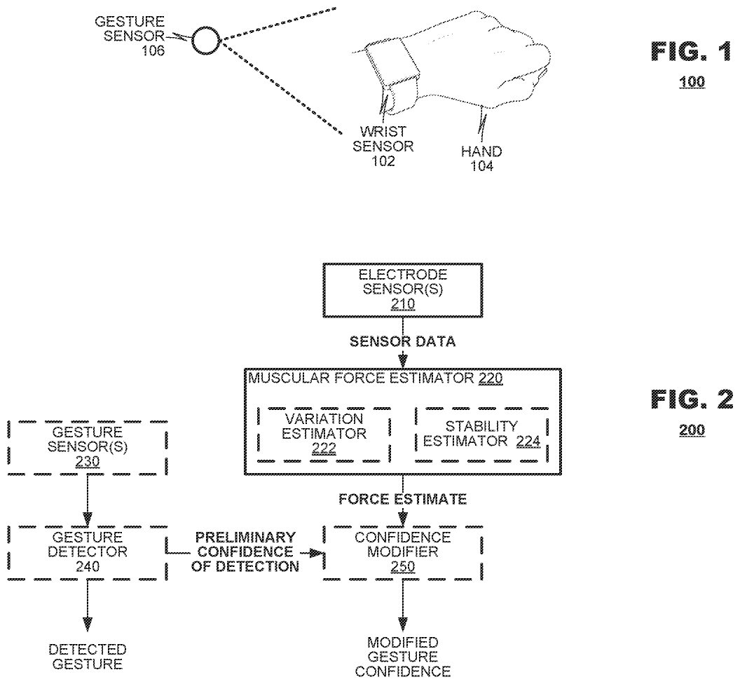

FIG. 1 illustrates an example system 100 for gesture recognition. System 100 includes a wrist sensor 102, attached to a subject user's hand 104, and also includes a gesture sensor 106 for capturing additional data regarding hand 104. In an aspect, wrist sensor 102 may include electrodes for measure a voltage at the surface of the skin of the user's wrist. In another aspect, gesture sensor 106 may be a camera capturing image of the user's hand 104.

While FIG. 1 depicts sensors for monitoring a hand, the disclosed techniques are not so limited. In aspects not depicted in FIG. 1, instead of a wrist, an electrode sensor may be attached to other parts of a user's body, such as a hand or other parts of an arm, leg, neck, or torso. In addition to sensing muscles of a hand, such a sensor may detect muscular force in other body parts, such as an arm, leg, or foot. Similarly, gesture sensor 106 may capture data regarding gestures performed by such other body parts.

In an aspect, gesture sensor 106 may be incorporated as part of headset worn by the subject user, or may be incorporated in a tablet, cell phone or other device positioned in proximity of the subject user and the user's gesturing body part (such as hand 104). Gesture sensor 106 may include a camera capable of capturing video or still images of visible light, infrared light, radar or sonar signals reflecting off the gesturing body part. In addition to or instead of a camera, gesture sensor 106 may include a motion sensor such as an accelerometer attached or coupled to the gesturing body part and may include one or more or other types of sensors for capturing data indicative of a gesture by a body part.

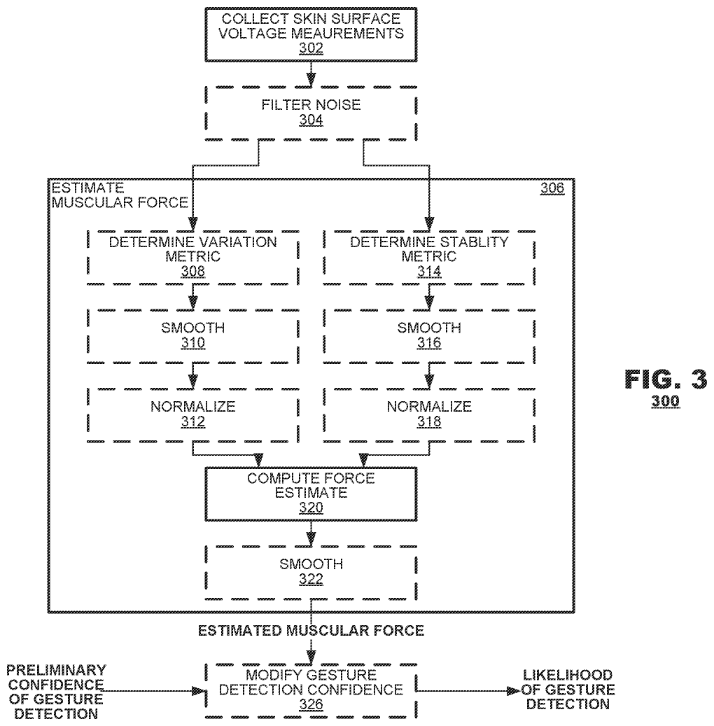

FIG. 2 illustrates an example system 200 for estimating muscular force. System 200 may be implemented, for example, in a device containing wrist sensor 102 of FIG. 1. System 200 includes an electrode sensor 210 and a muscular force estimator 220. In an aspect, electrode sensor 210 may be an electrode attached to the surface of a user's skin. In operation, electrode sensors 210 may provide a series of voltage measurements over time, and then muscular force estimator 220 may estimate, based on the voltage measurements, a muscular force of muscles inside the skin in a proximate area of the user's body adjacent to the electrode.

In an aspect, muscular force estimator 220 may include an estimator of signal variation 222 and may include an estimator of stability 224. In an aspect, the muscular force estimator may estimate a force based on a combination of variation metrics of the voltage measurements and stability of the voltage measurements. Additional details regarding estimation of muscular force are provided below regarding FIG. 3.

In an aspect, system 200 may use an estimate of muscular force to improve a recognition of gestures by a body part such as hand 104 (FIG. 1). The muscular force estimate may be related to gestures performed by a body part near the placement location of electrode sensor 210. For example, a force estimate from measurements at a wrist may be related to gestures performed by a hand connected to the wrist. Gesture detection may be improved, for example, by optional confidence modifier 250, which may modify a preliminary confidence estimate of a gesture detection based on a muscular force estimate. In an aspect, confidence modifier 250 may increase a preliminary confidence when an estimated muscular force is strong, and may decrease an preliminary confidence when the estimated muscular force is weak. For example, confidence modifier 250 may produce a modified gesture confidence by scaling the preliminary confidence by a magnitude of the muscular force estimate.

Additional optional aspects of system 200 may include gesture sensor(s) 230 and gesture detector 240. In an example based on FIG. 1, an electrode sensor 210 may be positioned on a wrist of the subject user, skeletal muscles that control the subject user's hand may affect the voltage measured by the proximate electrode sensor 210. In an aspect, electrode sensor 210 may be a differential pair of electrodes. A separate gesture sensor 230 for scanning gestures may be used by gesture detector 240 to: 1) detected a gesture of the hand; and 2) estimate a corresponding preliminary confidence in the gesture detection. A muscular force produced by muscular force estimator 220 may be used in combination with the preliminary confidence by confidence modifier 250 to produce a modified gesture confidence. For example, if the modified gesture confidence is below a threshold, a gesture detected by gesture detector 240 may be ignored or not passed on to a consumer of detected gestures.

In other aspects not depicted, muscular force estimator 220 may not be embodied in the same device as electrode sensor 210. For example, muscular force estimator may be incorporated in a device that also includes gesture sensors 106/230. Alternately, the muscular force estimator 220 may be included in a device that also include gesture detector 240, such as a cloud computer or cell phone that is paired with sensors 210, 230. One of skill in the art will understand that various other configurations are possible.

FIG. 3 illustrates an example process 300 for estimating muscular force. Process 300 includes collecting voltage measurement near the skin surface of a subject user (box 302). A muscular force may be estimated (box 306) for skeletal muscles of the subject user by computing the force estimate (box 320) based on the voltage measurements. In some optional aspects of process 300, a noise filter (box 304) may be applied to the voltage measurements, and the computed force estimate may be smoothed (box 322). In some implementations, a variation metric of the voltage measurements may be determined (308), and/or stability of the voltage measurements may be determined (314). In an aspect, a muscular force may be computed (box 320) as a compound metric based on the variation metric (from box 308), the stability metric (from box 314), and/or estimates of spectral properties of the voltage measurements (not depicted in FIG. 3).

A variation metric of the voltage measurements may be determined (box 308), for example, as a difference absolute standard deviation value (DASDV), which may be a standard deviation value of the difference between adjacent samples, such as:

$$D A S D V = \sqrt{\frac{1}{N - 1} {\sum }_{i = 1}^{N - 1} {( {x}_{i + 1} - {x}_{i} )}^{2}} , \qquad ( Eq . 1 )$$

where N is an integer window size of the voltage measurement samples x, and xᵢ refers to the i^{th} sample within the window. In another aspect, a variation metric may be determined as a median absolute deviation (MAD), which may be the median absolute difference between adjacent samples and their median or mean voltage, such as:

MAD=median(abs(xᵢ−median(x)))

(Eq. 2), where xᵢ refers to the i^{th} voltage measurement within a window of length N.

The determined variation (box 308) may be smoothed (box 310) and/or normalized (box 312) before being used to compute the force estimate (box 320). Smoothing of variation may be performed, for example, with a non-zero window size (box 310), and normalization (box 312) may be to a range from zero to 1.

In another aspect, the determined variation may be combined with a determined metric of stability in the series of voltage measurements. For example, a fractal dimension estimate (e.g., as computed with a method proposed by M. J. Katz) may indicate how detail in a pattern in the series of voltage measurements changes with the scale at which the pattern is measured:

$$Katz F D = \frac{log ( L / a )}{log ( d / a )} , \qquad ( Eq . 3 )$$

where the estimated fractal dimension is based on a set of sequential voltage measurement samples using a sum (L) and average (a) of the Euclidean distances between successive samples in the set, and using a maximum distance (d) between a first sample and all other samples in the set.

In an aspect, muscular force may be computed (box 320) by combining smoothed (boxes 310, 316) and/or normalized (boxes 312, 318) versions of the variation, spectral properties, and/or stability metric. Furthermore, the computed muscular force (box 320) may be further smoothed (box 322), such as with a non-zero length window.

In an aspect, smoothing, such as in optional boxes 310, 316, 322, may include techniques to remove noise, slow a rate of change, reduce high frequencies, or average over multiple neighboring samples. For example, smoothing operations may process a predetermined number of input samples to determine a single output sample, where a “window size” for the smoothing is the predetermined number. In as aspect, smoothing operations may differ between boxes 310, 316, and 322, and a corresponding window size for each may differ.

In aspects, a variety of normalization functions may be used. For example, a fixed normalization may be done using a fixed minimum and maximum, where the fixed minimum and fixed maximum are determined experimentally by a user. In other examples, normalization may be based a minimum and maximum over a window of sampled voltage measurement, where minimum and maximum are, for example, mean-based, median-based, or range-based. A mean-based normalization may have: minimum=mean−standard deviation*a factor; and maximum=mean+standard deviation*a factor. A median-based normalization may have: minimum=median−MAD*a factor; and maximum=median+MAD*a factor, where MAD is a median absolute deviation, as described above.

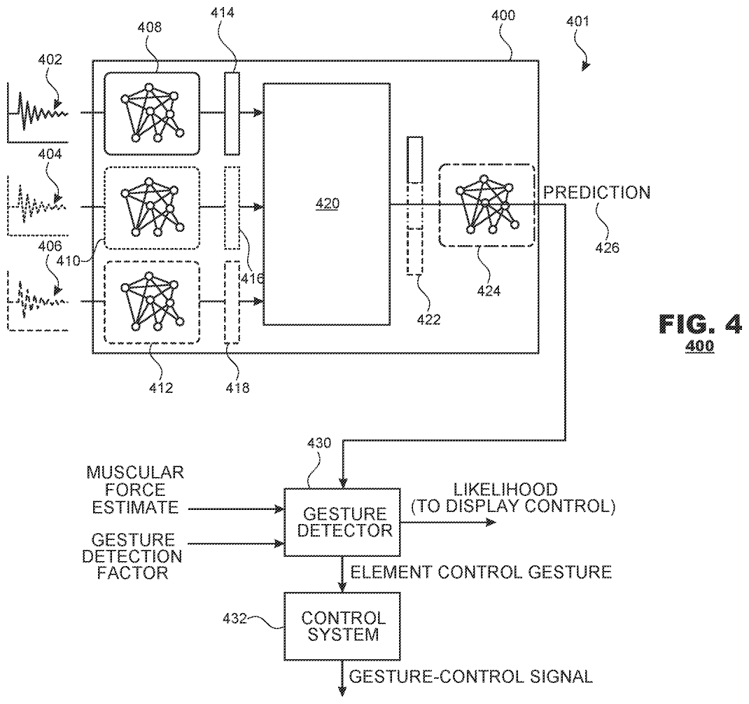

In an optional aspect of process 300, and a preliminary confidence of a gesture detection may be modified (box 326) based on an estimated muscular force to produce a likelihood of a detecting a gesture. A preliminary confidence of gesture detection may be, for example, an estimated probability that the subject user intended a particular gesture. See discussion below regarding gesture detector 430 (FIG. 4).

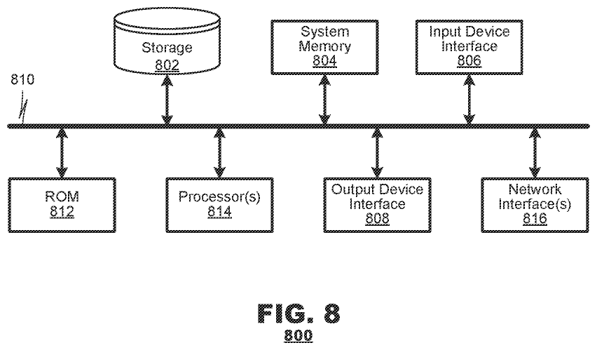

FIG. 4 illustrates schematic diagram of a gesture control performing a process for gesture control, in accordance with aspects of the disclosure. As shown in FIG. 4, sensor data from one or more sensors may be provided to gesture control system 401 (e.g., operating at the wrist sensor 102 (FIG. 1), system 200 (FIG. 2), or processor 814 (FIG. 8)). For example, the sensor data may include sensor data 402 (e.g., accelerometer data from one or more accelerometers), sensor data 404 (e.g., gyroscope data from one or more gyroscopes), and/or sensor data 406 from one or more physiological sensors (e.g., EMG data from an EMG sensor). As shown, the gesture control system 401 may include a machine learning system 400, a gesture detector 430, and/or a control system 432. In one or more implementations, the machine learning system 400, the gesture detector 430, and the control system 432 may be implemented at the same device, which may be the device in which the sensors that generate the sensor data is disposed, or may be a different device from the device in which the sensors that generate the sensor data are disposed. In one or more other implementations, the machine learning system 400, the gesture detector 430, and the control system 432 across multiple different devices, which may be include or be separate from the device in which the sensors that generate the sensor data are disposed. For example, the machine learning system 400 and the gesture detector 430 may be implemented at one device and the control system 432 may be implemented at a different device.

In one or more implementations, one or more of the sensor data 402, the sensor data 404, and the sensor data 406 may have characteristics (e.g., noise characteristics) that significantly differ from the characteristics of others of the sensor data 402, the sensor data 404, and the sensor data 406. For example, EMG data (e.g., sensor data 406) is susceptible to various sources of noise arising from nearby electrical devices, or bad skin-to-electrode contact. Therefore, EMG can be significantly noisier than accelerometer data (e.g., sensor data 402) or gyroscope data (e.g., sensor data 404). This can be problematic for training a machine learning model to detect a gesture based on these multiple different types of data with differing characteristics.

The system of FIG. 4 addresses this difficultly with multi-modal sensor data by, for example, providing the sensor data from each sensor to a respective machine learning model trained on sensor data of the same type. Intermediate processing operations 420 may also be performed to enhance the effectiveness of using multi-modal sensor data for gesture control. In the example of FIG. 4, sensor data 402 is provided as an input to a machine learning model 408, sensor data 404 is provided as an input to a machine learning model 410, and sensor data 406 is provided as an input to a machine learning model 412. In one or more implementations, machine learning model 408, machine learning model 410, and machine learning model 412 may be implemented as trained convolutional neural networks, or other types of neural networks.

For example, the machine learning model 408 may be a feature extractor trained to extract features of sensor data of the same type as sensor data 402, the machine learning model 410 may be a feature extractor trained to extract features of sensor data of the same type as sensor data 404, and the machine learning model 412 may be a feature extractor trained to extract features of sensor data of the same type as sensor data 406. As shown, machine learning model 408 may output a feature vector 414 containing features extracted from sensor data 402, machine learning model 410 may output a feature vector 416 containing features extracted from sensor data 404, and machine learning model 408 may output a feature vector 418 containing features extracted from sensor data 406. In this example, three types of sensor data are provided to three feature extractors, however, more or less than three types of sensor data may be used in conjunction with more or less than three corresponding feature extractors in other implementations.

As shown in FIG. 4, the feature vector 414, the feature vector 416, and the feature vector 418 may be processed in the intermediate processing operations 420 of the machine learning system 400 to combine aspects of the feature vector 414, the feature vector 416, and the feature vector 418 to generate a combined input vector 422 for input to a gesture prediction model 424.

In order to generate the combined input vector 422 for the gesture prediction model 424, the intermediate processing operations 420 may perform modality dropout operations, average pooling operations, modality fusion operations and/or other intermediate processing operations. For example, the modality dropout operations may periodically and temporarily replace one, some, or all of the feature vector 414, the feature vector 416, or the feature vector 418 with replacement data (e.g., zeros) while leaving the others of the feature vector 414, the feature vector 416, or the feature vector 418 unchanged. In this way, the modality dropout operations can prevent the gesture prediction model from learning to ignore sensor data from one or more of the sensors (e.g., by learning to ignore, for example, high noise data when other sensor data is low noise data). Modality dropout operations can be performed during training of the gesture prediction model 424, and/or during prediction operations with the gesture prediction model 424. In one or more implementations, the modality dropout operations can improve the ability of the machine learning system 400 to generate reliable and accurate gesture predictions using multi-mode sensor data. In one or more implementations, the average pooling operations may include determining one or more averages (or other mathematical combinations, such as medians) for one or more portions of the feature vector 414, the feature vector 416, and/or the feature vector 418 (e.g., to downsample one or more of the feature vector 414, the feature vector 416, and/or the feature vector 418 to a common size with the others of the feature vector 414, the feature vector 416, and/or the feature vector 418, for combination by the modality fusion operations). In one or more implementations, the modality fusion operations may include combining (e.g., concatenating) the features vectors processed by the modality dropout operations and the average pooling operations to form the combined input vector 422.

The gesture prediction model 424 may be a machine learning model that has been trained to predict a gesture that is about to be performed or that is being performed by a user, based on a combined input vector 422 that is derived from multi-modal sensor data. In one or more implementations, the machine learning system 400 of the gesture control system 401 (e.g., including the machine learning model 408, the machine learning model 410, the machine learning model 412, and the gesture prediction model 424) may be trained on sensor data obtained by the device in which the machine learning system 400 is implemented and from the user of that device, and/or sensor data obtained from multiple (e.g., hundreds, thousands, millions) of devices from multiple (e.g., hundreds, thousands, millions) of anonymized users, obtained with the explicit permission of the users. In one or more implementations, the gesture prediction model 424 may output a prediction 426. In one or more implementations, the prediction 426 may include one or more predicted gestures (e.g., of one or multiple gestures that the model has been trained to detect), and may also output a probability that the predicted gesture has been detected. In one or more implementations, the gesture prediction model may output multiple predicted gestures with multiple corresponding probabilities. In one or more implementations, the machine learning system 400 can generate a new prediction 426 based on new sensor data periodically (e.g., once per second, ten times per second, hundreds of times per second, once per millisecond, or with any other suitable periodic rate).

As shown in FIG. 4, the prediction 426 (e.g., one or more predicted gestures and/or one or more corresponding probabilities) from the gesture prediction model 424 may be provided to a gesture detector 430 (e.g., operating at the wrist sensor 102 (FIG. 1), system 200 (FIG. 2), or processor 814 (FIG. 8)). In one or more implementations, the gesture detector 430 may determine, a likelihood of a particular gesture (e.g., an element control gesture) being performed by the user based on the predicted gesture and the corresponding probability from the gesture prediction model 424 and based on a gesture detection factor.

In an aspect, outputs of gesture detector 430 may be further based on an estimate of muscular force such as described above regarding FIGS. 1-3. Gesture detector 430 may modify a preliminary confidence of gesture detection based on an estimate of muscular force, as in box 326 (FIG. 3), in order to produce a likelihood for a particular gesture prediction 426. For example, gesture detector 430 may combine a probability from the gesture prediction model 424 with an estimate of muscular force from box 306 in FIG. 3 to produce a likelihood of a corresponding gesture prediction 426.

For example, the gesture detector 430 may periodically generate a dynamically updating likelihood of an element control gesture (e.g., a pinch-and-hold gesture), such as by generating a likelihood for each prediction 426 or for aggregated sets of predictions 426 (e.g., in implementations in which temporal smoothing is applied). For example, when an element control gesture is the highest probability gesture from the gesture prediction model 424, the gesture detector 430 may increase the likelihood of the element control gesture based on the probability of that gesture from the gesture prediction model 424 and based on the gesture detection factor. For example, the gesture detection factor may be a gesture-detection sensitivity threshold. In one or more implementations, the gesture-detection sensitivity threshold may be a user-controllable threshold that the user can change to set the sensitivity of activating gesture control to the user's desired level. In one or more implementations, the gesture detector 430 may increase the likelihood of the element control gesture based on the probability of that gesture from the gesture prediction model 424, and based on the gesture detection factor by increasing the likelihood by an amount corresponding to a higher of the probability of the element control gesture and a fraction (e.g., half) of the gesture-detection sensitivity threshold.

In a use case in which the element control gesture is not the gesture with the highest probability from the gesture prediction model 424 (e.g., the gesture prediction model 424 has output the element control gesture with a probability that is lower than the probability of another gesture predicted in the output of the gesture prediction model 424), the gesture detector 430 may decrease the likelihood of the element control gesture by an amount corresponding the probability of whichever gesture has the highest probability from the gesture prediction model 424 and a fraction (e.g., half) of the gesture-detection sensitivity threshold. In this way, the likelihood can be dynamically updated up or down based on the output of the gesture prediction model 424 and the gesture detection factor (e.g., the gesture-detection sensitivity threshold).

As each instance of this dynamically updating likelihood is generated, the likelihood (e.g., or an aggregated likelihood based on several recent instances of the dynamically updating likelihood, in implementations in which temporal smoothing is used) may be compared to the gesture-detection sensitivity threshold. When the likelihood is greater than or equal to the gesture-detection sensitivity threshold, the gesture detector 430 may determine that the gesture has been detected and may provide an indication of the detected element control gesture to a control system 432. When the likelihood is less than the gesture-detection sensitivity threshold, the gesture detector 430 may determine that the gesture has not been detected and may not provide an indication of the detected element control gesture to a control system 432. In one or more implementations, providing the indication of the detected element control gesture may activate gesture-based control of an element at an electronic device (e.g., the wrist sensor 102 (FIG. 1), system 200 (FIG. 2), or processor 814 (FIG. 8) or another electronic device).

Throughout the dynamic updating of the likelihood by the gesture detector 430, the dynamically updating likelihood may be provided to a display controller. For example, the display controller (e.g., an application-level or system-level process with the capability of controlling display content for display operating at the wrist sensor 102 (FIG. 1), system 200 (FIG. 2), or device 800 (FIG. 8)) may generate and/or update a visual indicator. As the likelihood increases and decreases (and while the likelihood remains below the gesture-detection sensitivity threshold), the display controller may increase and decrease the overall size of the visual indicator, and/or may decrease and increase variability (variance) of one or more component sizes of one or more components of the visual indicator. When the element control gesture is provided to the control system 432 (e.g., responsive to the likelihood of the element control gesture reaching the threshold), this may coincide with the display controller increasing the visual indicator to its maximum size, changing its color, and/or animating the visual indicator to indicate activation of gesture control.

In various implementations, the control system 432 and/or the display controller may be implemented as, or as part of, a system-level process at an electronic device or as, or as part of an application (e.g., a media player application that controls playback of audio and/or video content, or a connected home application that controls smart appliances, light sources, or the like). In various implementations, the display controller may be implemented at the electronic device with the gesture prediction model 424 and the gesture detector 430 or may be implemented at a different device. In one or more implementations, the control system 432 and the display controller may be implemented separately or as part of a common system or application process.

Once the element control gesture is detected and the gesture-based control is activated, gesture control system 401 of FIG. 4 may continue to operate, such as to detect an ongoing hold of the element control gesture and/or a motion and/or rotation of the element control gesture. The gesture control system 401 may provide an indication of the motion and/or rotation to the control system 432 for control of the element (e.g., to rotate the virtual dial or slide the virtual slider).

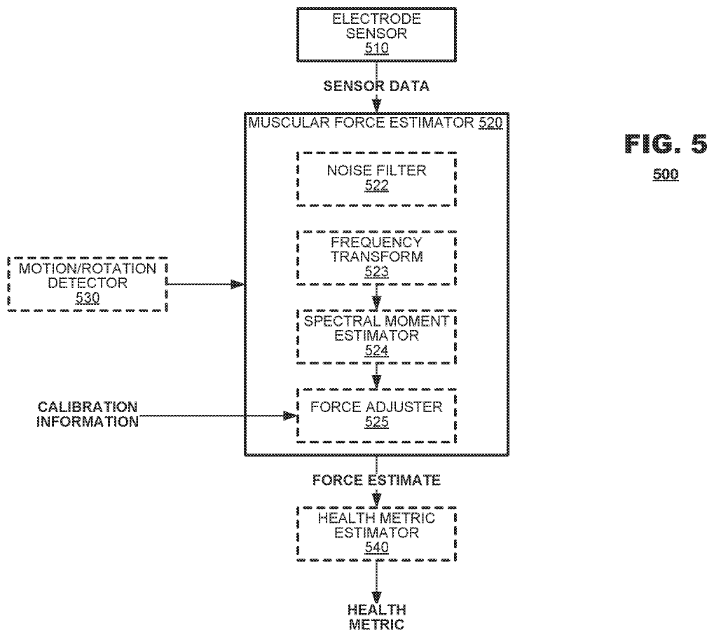

FIG. 5 illustrates an example system 500 for estimating muscular force. System 500 may be implemented, for example, in a device containing wrist sensor 102 of FIG. 1. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

System 500 includes an electrode sensor 510 and a muscular force estimator 520. In some implementations, some elements of system 500, such as any elements 520-540, may be implemented on a processor, such as processor 814 (FIG. 8). In an aspect, electrode sensor 510 may be an electrode attached to the surface of a user's skin. In operation, electrode sensors 510 may provide a series of voltage measurements over time, and then muscular force estimator 520 may estimate, based on the voltage measurements, a muscular force of muscles inside the skin in a proximate area of the user's body adjacent to the electrode.

In an aspect, muscular force estimator 520 may include spectral moment estimator 524. Spectral moment estimator may estimate a spectral moment of a series of voltage measurements from electrode sensor 510. A spectral moment may characterize a frequency spectrum of a series of measurements, and a first-order spectral moment may estimate a mean value of the frequency spectrum. Spectral moment estimator may determine a frequency spectrum of a series of measurements. Frequency transform 523 may transform a time-domain series of measurements, such as from the electrode sensor, into a frequency-domain representation. Frequency transform 523 may include, for example, a Fourier transform (such with a discrete Fourier transform (DFT), fast Fourier transform (FFT), or a discrete cosine transform (DCT)). In an aspect, the frequency-domain representation may include complex numbers each having a real and imaginary component.

In some implementations, a spectral moment may be computed as:

$$SpectralMoment = {\sum }_{k = 0}^{N - 1} k {P}_{k} \qquad ( Eq . 4 )$$

$${P}_{k} = \{ \begin{matrix}\frac{1}{2} log ( {re}_{k}^{2} + {im}_{k}^{2} ) , & if \sqrt{( {re}_{k}^{2} + {im}_{k}^{2} )} > 0 \\ 1 , & otherwise\end{matrix} ,$$

where:

N is the length of the signal,

k is the frequency index,

re_{f} is the real component of the frequency-domain representation of the frequency at index k, and

im_{f} is the imaginary component of frequency-domain representation of the frequency at index k.

In implementations, a series of electrode measurements from electrode sensor 510 may be filtered by noise filter 522 before calculating a muscular force. For example, noise filter 522 may include a high-pass filter for eliminating low frequency noise, and/or noise filter 522 may include a notch filter, for example to filter noise occurring around a particular notch frequency such as 60 Hz. Noise filter may be applied to a series of measurements prior to estimating a spectral moment, such as with spectral moment estimator 524.

In some implementations, an estimate of muscular force, such as from spectral moment estimator 524, may be adjusted by force adjuster 525 based on calibration information. For example, calibration information may indicate a correlation between an experimentally measured muscular force and an estimated spectral moment, and the calibration information may be used to “zero” adjust the muscular force estimate by shifting and/or scaling an estimated spectral moment to determine an estimated muscular force. In an aspect, calibration information may be determined based on a calibration process for electrode sensor 510 with a particular user. For example, a grip strength measuring device such as dynamometer may be held by the particular user in a hand that is also wearing the electrode sensor 510, and measurements during a calibration process may correlate dynamometer strength measurements with estimates of a spectral moment of electrode sensor measurements.

In an implementation a motion/rotation detector 530 may measure motion and/or rotation of electrode sensor 510, which may be used to disqualify muscular force estimates. For example, when motion or rotation of electrode sensor 510 is above respective thresholds, a muscular force estimate may be disqualified, or provided with an indication of low confidence. Large or fast motions or rotations of electrode sensor 510 may indicate movements of an arm electrode sensor 510, and the estimated muscular force may be unreliable at that time. For example, when an arm is moving, an estimated muscular force may in-part indicate forces of muscles used to move the arm and may not represent only force of muscles used for hand grip strength. In another aspect, an estimated muscular force may be disqualified whenever it is below a muscular force threshold.

Some health metrics may be based on estimates of muscular force. For example, a hand grip force estimate of a user from muscular force estimator 520 may be used by health metric estimator 540 to determine a health metric for the user. For example, a low grip strength or a fast drop in grip strength may be indicative of health problems.

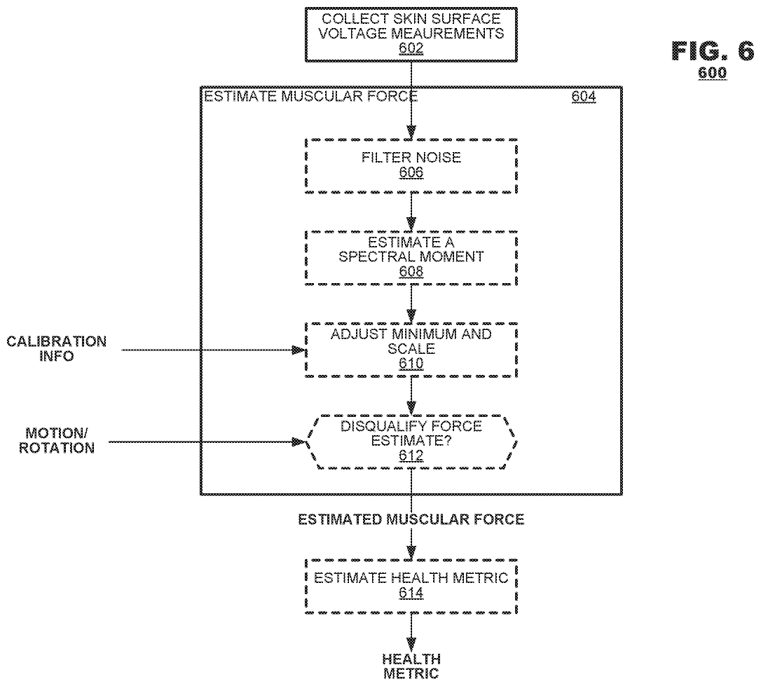

FIG. 6 illustrates an example process 600 for estimating muscular force. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

Process 600 may be implemented, for example, with system 500 (FIG. 5). Process 600 includes collecting voltage measurements near the skin surface of a subject user (602). A muscular force may be estimated (604), such as by muscular force estimator 720, for skeletal muscles of the subject user based on, for example, a spectral moment estimated from the voltage measurements (608). In some optional aspects of process 600, a noise filter may be applied (606) to the voltage measurements, such as by noise filter 522, prior to estimating the spectral moment (608). An estimated spectral moment may be adjusted according to calibration information (610), such as by force adjuster 525, for example by shifting and scaling an estimated spectral moment. Any resulting force estimates may be disqualified (612), based, for example, on motion and/or rotation information, such as from motion/rotation detector 530, or on a minimum threshold of estimated force. In an aspect, a force estimate may be used to estimate a health metric (614), such as by health metric estimator 540.

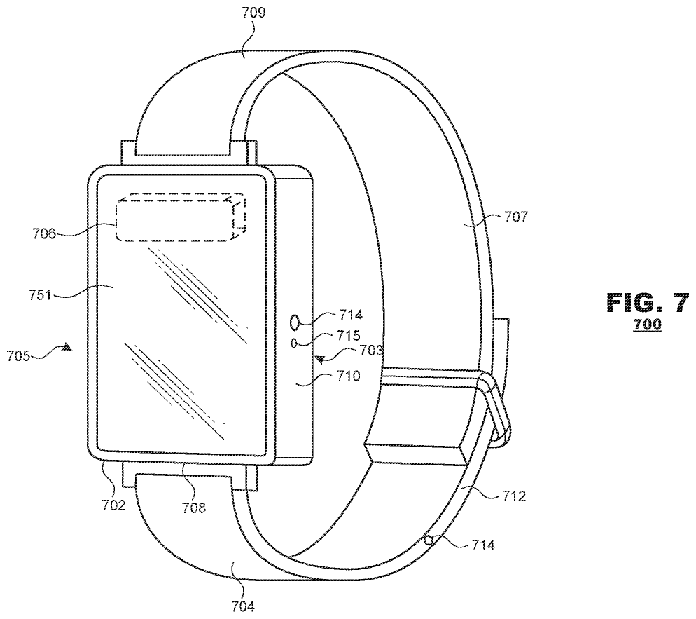

In one or more implementations, the system 200 and/or device 800 may include various sensors at various locations for determining proximity to one or more devices for gesture control, for determining relative or absolute locations of the device(s) for gesture control, and/or for detecting user gestures (e.g., by providing sensor data from the sensor(s) to machine learning a machine learning system). FIG. 7 illustrates an example electronic device 700 in which the system 200 or device 800 may be implemented in the form of the smart watch and may include wrist sensor 102 of FIG. 1, in one exemplary arrangement that can be used for gesture-based control of one or more electronic devices.

In the example of FIG. 7, electronic device 700 has been implemented in the form of a smartwatch. In this implementation, the electronic device 700 may be a standalone device that performs computing functions such as cellular telephone communications, WiFi communications, digital display functions, fitness tracking functions, or other computing functions, and/or may cooperate with one or more external devices or components such as a smartphone, a gaming system, or other computing system that is wirelessly paired or otherwise wirelessly coupled to the electronic device. For example, hand gestures performed by the hand on which the device is worn (e.g., on the attached wrist) can be used as input commands for controlling the electronic device 700 itself and/or for operating one or more other devices.

As shown in FIG. 7, the electronic device 700 may include a housing 702 and a band 704 that is attached to housing 702. In the example of FIG. 7, housing 702 forms a watch case having an outer surface 705 formed by a display 751. In this example, circuitry 706 (e.g., processor 814, system memory 804, sensors (e.g., 210, 230, or other sensors connected via input device interface 806), network interface 816 and/or other circuitry of the device 800 of FIG. 8) is disposed within the housing 702.

Housing 702 and band 704 may be attached together at interface 708. Interface 708 may be a purely mechanical interface or may include an electrical connector interface between circuitry within band 704 and circuitry 706 within housing 702 in various implementations. Processing circuitry such as the processor 814 of circuitry 706 may be communicatively coupled to one or more of sensors that are mounted in the housing 702 and/or one or more of sensors that are mounted in the band 704 (e.g., via interface 708).

In the example of FIG. 7, the housing 702 of the electronic device 700 includes sidewall 710 that faces the user's hand when the electronic device 700 is worn. In one or more implementations, the band 704 may also include a sidewall 712. Housing 702 also includes a wrist-interface surface 703 (indicated but not visible in FIG. 7) and an opposing outer surface 705 (e.g., formed by the display 751). Sidewall 710 extends between wrist-interface surface 703 and outer surface 705. In this example, band 704 includes a wrist-interface surface 707 and an opposing outer surface 709, and sidewall 712 extends between wrist-interface surface 707 and outer surface 709.

In one or more implementations, one or more of the sensors 210, 230 may be mounted on or to the sidewall 710 of housing 702. In the example of FIG. 7, an ultra-wide band (UWB) sensor 714 is provided at or near the sidewall 710. In the example of FIG. 7, the electronic device 700 also includes a camera 715 mounted in or to the sidewall. In the example of FIG. 7, the electronic device 700 also include a UWB sensor 714 at or near the sidewall 712 of the band 704. However, this is merely illustrative. In various implementations, a UWB sensor 714 may be provided on or within the housing 702 without any cameras on or within the housing 702, and/or without any cameras or UWB sensors in the band 704.

Although various examples, including the example of FIG. 7, are described herein in which a UWB sensor is used to determine a direction in which a device is pointing and/or another device at which the device is aimed or pointed, it is appreciated that other sensors and/or sensing technologies may be used for determining a pointing direction of a device and/or to recognize another device at which the device is aimed or pointed. As examples, other sensors and/or sensing technologies may include a computer-vision engine that receives images of the device environment from an image sensor, and/or a BLE sensor.

Although not visible in FIG. 7, one or more additional sensors 212 may also be provided on wrist-interface surface 703 of housing 702, and communicatively coupled with the circuitry 706. The additional sensors 212 that may be provided on wrist-interface surface 703 may include a photoplethysmography (PPG) sensor configured to detect blood volume changes in microvascular bed of tissue of a user (e.g., where the user is wearing the electronic device 700 on his/her body, such as his/her wrist). The PPG sensor may include one or more light-emitting diodes (LEDs) which emit light and a photodiode/photodetector (PD) which detects reflected light (e.g., light reflected from the wrist tissue). The additional sensors 212 that may be provided on wrist-interface surface 703 may additionally or alternatively correspond to one or more of: an electrocardiogram (ECG) sensor, an electromyography (EMG) sensor, a mechanomyogram (MMG) sensor, a galvanic skin response (GSR) sensor, and/or other suitable sensor(s) configured to measure biosignals. In one or more implementations, the electronic device 700 may additionally or alternatively include non-biosignal sensor(s) such as one or more sensors for detecting device motion, sound, light, wind and/or other environmental conditions. For example, the non-biosignal sensor(s) may include one or more of: an accelerometer for detecting device acceleration, rotation, and/or orientation, one or more gyroscopes for detecting device rotation and/or orientation, an audio sensor (e.g., microphone) for detecting sound, an optical sensor for detecting light, and/or other suitable sensor(s) configured to output signals indicating device state and/or environmental conditions, and may be included in the circuitry 706.

FIG. 8 illustrates an example computing device 800 with which aspects of the subject technology may be implemented in accordance with one or more implementations. For example, computing device 800 may be used for performing process 300 (FIG. 3), may be used for performing the process 600 (FIG. 6), may be used for implementing one or more components of example systems 200 (FIG. 2) or 500 (FIG. 5), and may be used for implementing the example process and system of FIG. 4. The computing device 800 can be, and/or can be a part of, any computing device or server for generating the features and processes described above, including but not limited to a laptop computer, a smartphone, a tablet device, a wearable device such as a goggles or glasses, an earbud or other audio device, a case for an audio device, and the like. The computing device 800 may include various types of computer readable media and interfaces for various other types of computer readable media. The computing device 800 includes a permanent storage device 802, a system memory 804 (and/or buffer), an input device interface 806, an output device interface 808, a bus 810, a ROM 812, one or more processors 814, one or more network interface(s) 816, and/or subsets and variations thereof.

The bus 810 collectively represents all system, peripheral, and chipset buses that communicatively connect the numerous internal devices of the computing device 800. In one or more implementations, the bus 810 communicatively connects the one or more processors 814 with the ROM 812, the system memory 804, and the permanent storage device 802. From these various memory units, the one or more processors 814 retrieves instructions to execute and data to process in order to execute the processes of the subject disclosure. The one or more processors 814 can be a single processor or a multi-core processor in different implementations.

The ROM 812 stores static data and instructions that are needed by the one or more processors 814 and other modules of the computing device 800. The permanent storage device 802, on the other hand, may be a read-and-write memory device. The permanent storage device 802 may be a non-volatile memory unit that stores instructions and data even when the computing device 800 is off. In one or more implementations, a mass-storage device (such as a magnetic or optical disk and its corresponding disk drive) may be used as the permanent storage device 802.

In one or more implementations, a removable storage device (such as a floppy disk, flash drive, and its corresponding disk drive) may be used as the permanent storage device 802. Like the permanent storage device 802, the system memory 804 may be a read-and-write memory device. However, unlike the permanent storage device 802, the system memory 804 may be a volatile read-and-write memory, such as random-access memory. The system memory 804 may store any of the instructions and data that one or more processors 814 may need at runtime. In one or more implementations, the processes of the subject disclosure are stored in the system memory 804, the permanent storage device 802, and/or the ROM 812. From these various memory units, the one or more processors 814 retrieves instructions to execute and data to process in order to execute the processes of one or more implementations.

The bus 810 also connects to the input and output device interfaces 806 and 808. The input device interface 806 enables a user to communicate information and select commands to the computing device 800. Input devices that may be used with the input device interface 806 may include, for example, alphanumeric keyboards and pointing devices (also called “cursor control devices”). The output device interface 808 may enable, for example, the display of images generated by computing device 800. Output devices that may be used with the output device interface 808 may include, for example, printers and display devices, such as a liquid crystal display (LCD), a light emitting diode (LED) display, an organic light emitting diode (OLED) display, a flexible display, a flat panel display, a solid-state display, a projector, or any other device for outputting information.

One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 8, the bus 810 also couples the computing device 800 to one or more networks and/or to one or more network nodes through the one or more network interface(s) 816. In this manner, the computing device 800 can be a part of a network of computers (such as a LAN, a wide area network (“WAN”), or an Intranet, or a network of networks, such as the Internet. Any or all components of the computing device 800 can be used in conjunction with the subject disclosure.

In one or more implementations, the system memory 804 may store one or more feature extraction models, one or more gesture prediction models, one or more gesture detectors, one or more (e.g., virtual) controllers (e.g., sets of gestures and corresponding actions to be performed by the device 800 or another electronic devices when specific gestures are detected), voice assistant applications, and/or other information (e.g., locations, identifiers, location information, etc.) associated with one or more other devices, using data stored locally in system memory 804. Moreover, the input device 806 may include suitable logic, circuitry, and/or code for capturing input, such as audio input, remote control input, touchscreen input, keyboard input, etc. The output device interface 808 may include suitable logic, circuitry, and/or code for generating output, such as audio output, display output, light output, and/or haptic and/or other tactile output (e.g., vibrations, taps, etc.).

The sensors included in or connected to input device interface 806 may include one or more ultra-wide band (UWB) sensors, one or more inertial measurement unit (IMU) sensors (e.g., one or more accelerometers, one or more gyroscopes, one or more compasses and/or magnetometers, etc.), one or more image sensors (e.g., coupled with and/or including an computer-vision engine), one or more electromyography (EMG) sensors, optical sensors, light sensors, image sensors, pressure sensors, strain gauges, lidar sensors, proximity sensors, ultrasound sensors, radio-frequency (RF) sensors, platinum optical intensity sensors, and/or other sensors for sensing aspects of the environment around and/or in contact with the device 800 (e.g., including objects, devices, and/or user movements and/or gestures in the environment). The sensors may also include motion sensors, such as inertial measurement unit (IMU) sensors (e.g., one or more accelerometers, one or more gyroscopes, and/or one or more magnetometers) that sense the motion of the device 800 itself.

In one or more implementations, system memory 804 may store a machine learning system that includes one or more machine learning models that may receive, as inputs, outputs from one or more of sensor(s) (e.g. sensors 210, 230 which may be connected to input device interface 806). The machine learning models may have been trained based on outputs from various sensors corresponding to the sensors(s), in order to detect and/or predict a user gesture. When the device 800 detects a user gesture using the sensor(s) and the machine learning models, the device 800 may perform a particular action (e.g., raising or lowering a volume of audio output being generated by the device 800, scrolling through video or audio content at the device 800, other actions at the device 800, and/or generating a control signal corresponding to a selected device and/or a selected gesture-control element for the selected device, and transmitting the control signal to the selected device). In one or more implementations, the machine learning models may be trained based on a local sensor data from the sensor(s) at the device 800, and/or based on a general population of devices and/or users. In this manner, the machine learning models can be re-used across multiple different users even without a priori knowledge of any particular characteristics of the individual users in one or more implementations. In one or more implementations, a model trained on a general population of users can later be tuned or personalized for a specific user of a device such as the device 800.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components (e.g., computer program products) and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to,” “operable to,” and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration.” Any embodiment described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A device, comprising:
a differential pair of electrodes configured to provide voltage measurements at a single location on a user; and
circuitry configured to estimate a muscular force by:
collecting a series of the voltage measurements from the electrodes over time; and
estimating a muscular force based on the series of the voltage measurements from the electrodes.

2. The device of claim 1, wherein the single location on the user includes a wrist of the user.

3. The device of claim 1, wherein the circuitry is further configured to estimate a health metric of the user based on the estimated muscular force.

4. The device of claim 1, wherein the estimating the muscular force includes deriving a spectral moment of the series of the voltage measurements, and the estimated muscular force is based on the spectral moment.

5. The device of claim 4, wherein the spectral moment is a first spectral moment within a window of the series of voltage measurements.

6. The device of claim 4, wherein the estimating the muscular force further includes adjusting the spectral moment based on a minimum spectral moment, and wherein the estimated muscular force is based the adjusted spectral moment and the minimum spectral moment is based on a calibration of the user with the electrodes.

7. The device of claim 4, wherein the estimating the muscular force includes filtering the series of voltage measurements with a high-pass filter, and wherein the spectral moment is based on the high-pass filtered voltage measurements.

8. The device of claim 4, wherein the estimating the muscular force includes computing a frequency response over a window of the series of voltage measurements, and wherein the spectral moment is a sum over frequencies in the frequency response of a summation frequency times a logarithm of the frequency response at the summation frequency.

9. The device of claim 1, wherein voltage measurements are discarded when one or more of the following conditions occur: an acceleration of the electrodes is above a threshold; a rotation of the electrodes is above a threshold; and the estimated muscular force is below a threshold.

10. The device of claim 1, wherein the estimating muscular force includes deriving a metric of variation of adjacent voltage measurements within a window of the series of the voltage measurements, and the estimated muscular force is based on the metric of variation.

11. The device of claim 1, wherein the estimating muscular force includes estimating a fractal dimension of the voltage measurements, and the estimated muscular force is based on the fractal dimension.

12. The device of claim 1, wherein the estimating muscular force includes:
deriving a metric of variation of voltage measurements within a window of the series of the voltage measurements;
estimating a metric of stability of the voltage measurements; and
the estimated muscular force is based on a combination of the metric of variation and the metric of stability.

13. The device of claim 12, wherein the estimating muscular force further includes:
smoothing the metric of variation with a non-zero window size;
normalizing the smoothed metric of variation;
smoothing the metric of stability with a non-zero window size;
normalizing the smoothed metric of stability;
smoothing the estimated muscular force with a non-zero window size.

14. A method, comprising:
collecting a series of voltage measurements over time from electrodes attached to a wrist of a user; and
estimating a muscular force based on the series of voltage measurements from the electrode.

15. The method of claim 14, wherein the electrodes are a differential pair of electrodes for measuring voltage at a single location on the user, and the estimating the muscular force is based on voltage measurements from the differential pair when attached to the wrist of the user.

16. The method of claim 14, further comprising estimating a health metric of the user based on the estimated muscular force.

17. The method of claim 14, wherein the estimating the muscular force includes deriving a spectral moment of the series of the voltage measurements, and the estimated muscular force is based on the spectral moment.

18. The method of claim 17, wherein the spectral moment is a first spectral moment within a window of the series of voltage measurements.

19. The method of claim 17, wherein the estimating the muscular force further includes adjusting the spectral moment based on a minimum spectral moment, and wherein the estimated muscular force is based the adjusted spectral moment and the minimum spectral moment is based on a calibration of the user with the electrode.

20. The method of claim 17, wherein the estimating the muscular force includes filtering the series of voltage measurements with a high-pass filter, and wherein the spectral moment is based on the high-pass filtered voltage measurements.

21. The method of claim 17, wherein the estimating the muscular force includes computing a frequency response over a window of the series of voltage measurements, and wherein the spectral moment is a sum over frequencies in the frequency response.

22. The method of claim 14, wherein voltage measurements are discarded when one or more of the following conditions occur: an acceleration of the electrode is above a threshold; a rotation of the electrode is above a threshold; and the estimated muscular force is below a threshold.

23. A non-transitory computer readable medium storing instructions that, when executed by a processor, cause the process to:
collecting a series of voltage measurements over time from an electrode attached to a wrist of a user; and
estimating a muscular force based on the series of voltage measurements.

24. The non-transitory computer readable medium of claim 23, wherein the electrodes are a differential pair of electrodes for measuring voltage at a single location on the user, and the estimating the muscular force is based on voltage measurements from the differential pair of electrodes when attached to the wrist of the user.
